(19) United States
(12) Patent Application Publication
(10) Pub. No.: US 2007/0232883 A1
(43) Pub. Date: Oct. 4, 2007

(54) Systems and methods for determining plaque vulnerability to rupture

(76) Inventor: Olusegun Johnson Ilegbusi, Oviedo, FL (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/699,935

(22) Filed: Jan. 30, 2007

Related U.S. Application Data
(63) Continuation-in-part of application No. 11/494,299, filed on Jul. 27, 2006, now abandoned.
Application No. 11/494,299 is a continuation of application No. 11/417,599, filed on May 4, 2006.
(60) Provisional application No. 60/773,486, filed on Feb. 15, 2006.

(51) Int. Cl. A61B 5/05

(52) U.S. Cl. 600/407

(57) ABSTRACT

In some embodiments, stress values for a diseased artery are obtained, stress ratios are determined from the obtained stress values, and a flow-structure interaction index is generated based upon the stress ratios as a function of a given plaque characteristic. In further embodiments, a plaque characteristic of a patient is determined, a patient's stress ratio is determined in relation to the flow-structure interaction index and the plaque characteristic, and the patient's stress ratio is compared to a critical stress ratio to determine whether the patient's stress ratio exceeds the critical stress ratio.

### CROSS-REFERENCE TO RELATED APPLICATIONS

This application is a continuation-in-part of U.S. non-provisional application Ser. No. 11/494,299, entitled, “Systems And Methods For Evaluating Vessels,” filed Jul. 27, 2006, which is a continuation of U.S. non-provisional application Ser. No. 11/417,599, entitled, “Optical Probes For Imaging Narrow Vessels Or Lumens,” filed May 4, 2006 which claims priority to U.S. provisional application Ser. No. 60/773,486, entitled, “Optical Apparatuses and Methods,” filed Feb. 15, 2006, each of which is hereby incorporated by reference in their entireties.

### BACKGROUND

Coronary artery diseases (CADs) are the leading cause of death in the developed world. They are referred to as “silent killers” given that they are often asymptomatic until the patient suffers a heart attack.

Plaque rupture with superimposed thrombosis is the primary cause of acute coronary syndromes of unstable angina, myocardial infarction, and sudden deaths. The transition into unstable plaques is normally characterized by the presence of active inflammation (monocyte/macrophage infiltration), thinning of the fibrous cap of the plaque, development of a large lipid necrotic core, and endothelial denudation with superficial platelet aggregation. Although such a condition is serious, it can be treated, at least in some cases, with aggressive therapy intended to prevent a catastrophic vascular event if the existence and location of the vulnerable plaque are detected.

Techniques currently exist that are used to detect unstable plaques and therefore diagnose possible plaque rupture. Unfortunately, unstable plaques that are at risk of rupture often may not be identified by such techniques for various reasons, including poor resolution of the imaging modality, slow system response, and the complexity of the plaques and the forces acting upon them. Thus, the practice of such techniques may not result in the detection of vulnerable plaques that, if otherwise detected, could be treated.

### SUMMARY

Disclosed are systems and methods for determining plaque vulnerability to rupture. In some embodiments, stress values for a diseased artery are obtained, stress ratios are determined from the obtained stress values, and a flow-structure interaction index is generated based upon the stress ratios as a function of a given plaque characteristic. In further embodiments, a plaque characteristic of a patient is determined, a patient stress ratio is determined in relation to the flow-structure interaction index and the plaque characteristic, and the patient's stress ratio is compared to a critical stress ratio to determine whether the patient's stress ratio exceeds the critical stress ratio.

### BRIEF DESCRIPTION OF THE FIGURES

The components in the drawings are not necessarily to scale, emphasis instead being placed upon clearly illustrating the principles of the present disclosure. In the drawings, like reference numerals designate corresponding parts throughout the several views.

FIG. 1 is a flow diagram of an embodiment of a method for generating a fluid-structure interaction index.

FIG. 2 illustrates an embodiment of a model of a diseased artery.

FIG. 3 is a bi-linear stress-strain curve.

FIG. 4 illustrates predicted flow patterns for a diseased artery.

FIG. 5 illustrates predicted shear stress distributions for a diseased artery exhibiting 20%, 40%, and 70% stenosis.

FIGS. 6A and 6B illustrate representative predicted stress contours for a diseased artery.

FIGS. 7A and 7B illustrate predicted maximum principal stress and Von Mises stress distributions for a diseased artery, for 20% stenosis and 70% stenosis, respectively.

FIGS. 8A and 8B illustrate maximum principal stress and circumferential stress distributions for a diseased artery, for 20% stenosis and 70% stenosis, respectively.

FIGS. 9A and 9B illustrate a first set of predicted stress ratios for a diseased artery, for 20% stenosis and 70% stenosis, respectively.

FIGS. 10A and 10B illustrate a second set of predicted stress ratios for a diseased artery, for 20% stenosis and 70% stenosis, respectively.

FIG. 11 illustrates fluid-structure interaction indices as a function of stenosis level.

FIG. 12 illustrates a fluid-structure interaction index as a function of fibrous cap thickness.

FIG. 13 illustrates a fluid-structure interaction index as a function of lipid pool location.

FIG. 14 illustrates fluid-structure interaction indices as a function of lipid pool volume and calcium volume relative to total plaque volume.

FIG. 15 illustrates stress ratio indices as a function of stenosis level for various blood pressures.

FIG. 16 is a flow diagram of an embodiment of a method for predicting the likelihood of plaque rupture.

FIG. 17 is a block diagram of an embodiment of a computer system that comprises logic configured to generate a fluid-structure interaction index.

### DETAILED DESCRIPTION

Introduction

As described above, current technologies may be ineffective in enabling identification of unstable arterial plaques that are prone to rupture. Given that such plaques could be treated if detected, it can be appreciated that there is a need for systems and methods that can be used to identify unstable plaques with high potential to rupture.

In the following, described are various embodiments of systems and methods for determining plaque vulnerability. As described below, the plaque potential to rupture can be determined by considering the nature of both the fluid flow through the artery and the structural characteristics of the plaque. In some embodiments, both shear stresses and structural stresses are considered in developing an index, designated as the flow-structure interaction (FSI) index, that is indicative of plaque potential to rupture. Through comparison of such an index and observed conditions of a patient under evaluation, a determination as to that patient's plaque potential to rupture can be made.

Although evaluation of coronary arteries is discussed in detail in this disclosure, it is to be appreciated that the disclosed systems and methods can be used to evaluate other arteries. In addition, the disclosed systems and methods may be used in conjunction with other body vessels, or other biological or non-biological vessels as the case may warrant. Furthermore, although particular embodiments of systems and methods are described in the following, those embodiments are mere example implementations of the systems and methods and it is noted that other embodiments are possible. All such embodiments are intended to be within the scope of this disclosure. The terminology used in this disclosure is selected for the purpose of describing the disclosed systems and methods and is not intended to limit the breadth of the disclosure.

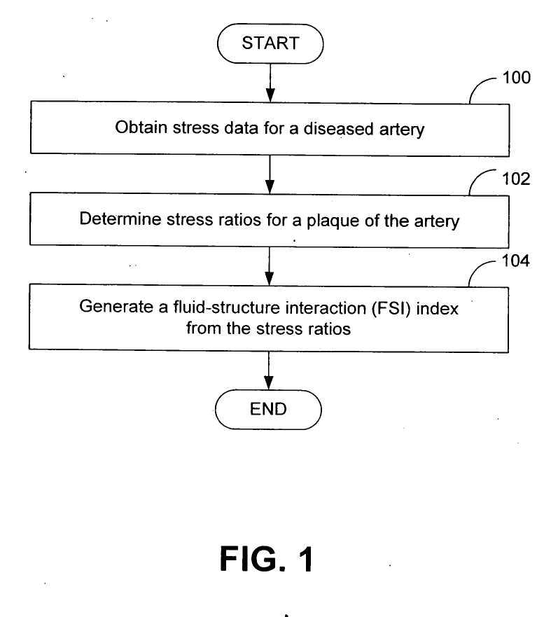

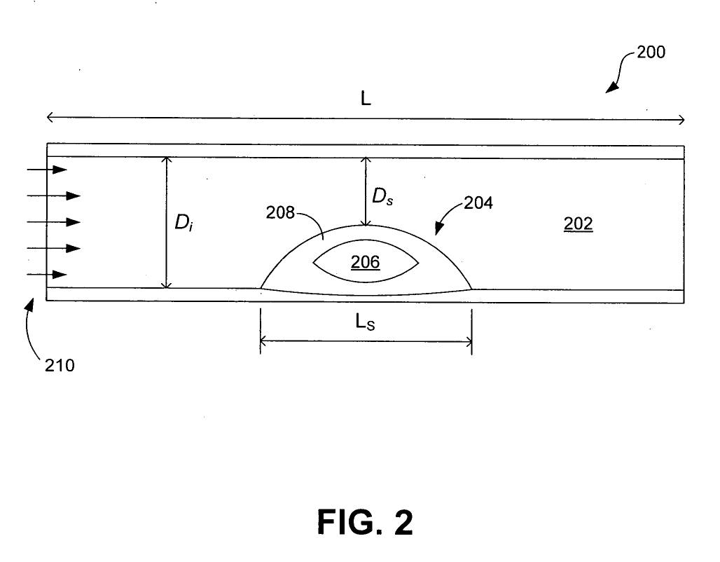

FIG. 1 provides an overview of a method for generating a flow-structure interaction (FSI) index based upon stresses that act on a plaque. Beginning with block 100, stress data for a diseased artery is obtained. As described in the following, various stress values pertinent to an arterial plaque can be considered. The stress values can be computed through computational modeling or through the collection of empirical data. Methods for determining the stress values in the former case are described in detail in relation to FIGS. 2-10 below. In the latter case, the stress values can be measured or estimated through testing of human or animal subjects and/or through physical modeling of diseased arteries. In some cases, non-invasive optical techniques can be used to determine the stress values pertinent to an actual artery. At least in the case of animal testing, such stress values can be determined through to plaque rupture to provide the most relevant data as to the relationship between the stresses and plaque rupture.

It is noted that stress data can be obtained for a variety of subject conditions. For example, stress values can be obtained for each of several cases, including: normal blood pressure subjects, high blood pressure subjects, low blood pressure subjects, subjects who smoke, etc. By collecting such data, various FSI indices can be generated that are custom tailored for various types of patients that are to be evaluated. Furthermore, it is noted that stress values can be obtained relative to various plaque characteristics that may be encountered. For example, stress values can be obtained for varying levels of stenosis. In another example, stress values can be obtained in relation to the thickness of the plaque's fibrous cap, the location of the lipid core within the plaque, or the size of the lipid core, to name a few. In view of the above, stress data can be obtained for a variety of types of subjects exhibiting plaques having a variety of characteristics.

Turning to block 102, stress ratios for the plaque are determined. As described in the following, the stress ratios take into account stresses related to blood flow through the artery as well as stresses related to the structure of the plaque itself as both forms of stress are relevant to plaque rupture.

With reference next to block 104, the stress ratio data is used to generate at least one FSI index that can be used as a tool for determining plaque potential to rupture.

Determination of Flow and Structure Related Stresses

In the following, an example method of computing stress ratios through mathematical modeling is described. FIG. 2 illustrates a model of a segment of a diseased artery 200. The artery 200 comprises a channel or lumen 202 in which a stenosis or plaque 204 exists. A small lipid core 206, primarily comprising cholesterol, is embedded within the plaque 204 under a fibrous cap 208 of the plaque. In the figure, L and Ls represent the lengths of the artery 200 and the plaque 204, respectively. The percent stenosis by diameter Sₜ is defined as follows:

$${S}_{t} = \frac{{D}_{i} - {D}_{s}}{{D}_{i}} \times 100 ( \% ) \qquad [ Equation 1 ]$$

where Dᵢ and Dₛ are, respectively, the nominal diameter and the minimum diameter of the artery lumen 202, respectively. As shown in FIG. 2, blood flow through the artery 200 is assumed to be from the left to the right, as indicated by flow arrows 210.

Stenosis volume or severity is not the largest determinant of plaque tearing. Studies have shown that less obstructive plaques are more prone to rupture than larger plaques and that plaque tearing is more closely related to stress concentrations resulting from hemodynamic and biomechanical forces acting on the plaque. Therefore, by investigating the correlation between different stages of plaque formation and patterns of stress, unstable plaques that are prone to rupture can be identified and treated before they rupture. In FIG. 2, the example artery model 100 is assumed to have the following geometry: L=110 millimeters (mm), Ls=10 mm, Dᵢ=4 mm, and Dₛ=0.5 mm. The portion of the artery 200 upstream of the plaque 204 is chosen to be 50 mm long (10 Dᵢ), enabling flow development ahead of the plaque. A similar length is provided downstream of the plaque 204 to allow flow recovery before the outlet. The latter also ensures reliable outlet boundary condition in the computation of the fluid flow equations.

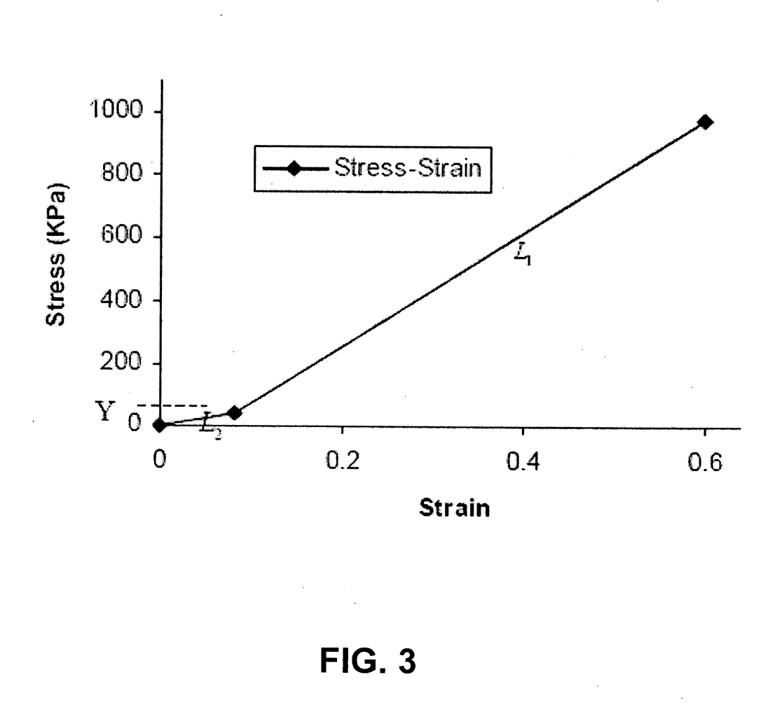

In the modeling, mild (20% stenosis), moderate (30%, 40%, and 50% stenosis), and severe (70% stenosis) cases are considered. The eccentricity is assumed to be 100% in all cases to reflect common diseased arteries. The model assumes bi-linear isotropic, incompressible material properties. Specifically, a bi-linear model is used, which is defined by the stress-strain curve and the two Young moduli, E₁ and E₂, for stress values that are respectively less than and greater than the yield stress Y. That particular model is used because it reflects an optimization scheme in the sense that the model provides a good approximation as to the non-linear behavior of the plaque under shear stress and internal pressure. In addition, the model is readily implemented in multi-purpose software for simulating fluid structure interactions. FIG. 3 presents a typical bilinear stress-strain curve. The slopes of the lines L₁ and L₂ provide two Young moduli, E₁ and E₂. The approximation of the non-linear stress-strain curve is completely defined by Y, E₁, and E₂.

Trilateral and quadrilateral finite elements are generated for the fluid and solid parts of the arterial segment, resulting in 8505 to 9354 elements and 7029 to 7683 nodes per model. Unlike previous studies, the internal luminal pressure is not prescribed but rather computed from the flow module and distributed over the inner surface. The input parameters used herein are summarized in Table 1:

TABLE 1
Inlet Velocity 0.2 m/s
Outlet Gage Pressure, 0 Pa

| | | | Modu- | | | |
|---|---|---|---|---|---|---|
| | Density | Kinematic | lus | Yield | | Poission's |
| | ρ- | Viscosity | E₁- | Stress | Modulus | Ratio |
| Materials | Kg/m³ | υ-m²/s | kN/m² | Y | E₂-kN/m² | θ |
| Blood-like | 1050 | 3.6 × 10⁻⁶ | | | | |
| Artery | | | 61.5 | 8.4 | 245 | 0.45 |
| Plaque | | | 483 | 39.6 | 1820 | 0.45 |
| Lipid core | | | 3.81 | 0.69 | 38.8 | 0.45 |

Previous studies have demonstrated the significant impact of endothelial shear stress and structural stresses on plaque rupture. In addition, the maximum principal stresses and Von Mises stresses have been predicted. By analogy to the concept of buckling in material failure study, the normalized wall shear stresses obtained from the flow model by each of the above structural stresses can be used for analysis of the potential of a plaque to rupture.

The following equilibrium and boundary conditions for the artery wall are used:
σ_{ij,j}^{(Sd)}=0 [Equation 2]
σᵢⱼ^{(Sd)}·nⱼ|inner surf=σᵢⱼ^{(fd)}·nⱼ|innersurf [Equation 3]
d^{(Sd)}inner surf|=d^{(fd)}|innersurf [Equation 4]
d_{—}_{^{Y}}^{(Sd)}|outersurf=0 [Equation 5]
d_{—}_{^{X}}^{(Sd)}|inlet,outersurf=0 [Equation 6]
where, d^{(Sd)}(d_{—}_{^{x,}}^{d}_{—}_{^{y}}^{s}), d^{(fd)} are the displacements (X and Y directions respectively) and σᵢⱼ^{(Sd)},σᵢⱼ^{(fd)} are the stress tensors for solid and fluid, respectively.

Steady, viscous, incompressible flow are assumed for the artery model and the fluid is assumed to be Newtonian. In other embodiments the fluid could be modeled as non-Newtonian without loss of the essential characteristics of the predicted results. The transport equations governing blood flow with compliant walls are solved, for example, using the CFD-ACE-GUI computer code available from EAI, Huntsville, Ala.

The governing equations for the steady flow behavior can be expressed as:

$$\begin{matrix}Flow direction \text{:} & \\ ∇ \cdot ( {V}^{r} u ) = \frac{1}{ρ} [ - \frac{\partial p}{\partial x} + ∇ \cdot ( μ ∇ u ) ] \\ Transverse direction \text{:} & [ Equation 7 ] \\ ∇ \cdot ( {V}^{r} u ) = \frac{1}{ρ} [ - \frac{\partial p}{\partial y} + ∇ \cdot ( μ ∇ u ) ] & [ Equation 8 ]\end{matrix}$$

In the above equations, p is the static pressure and τᵢⱼ is the viscous stress tensor.

For boundary conditions, it is assumed that there is no-slip on the arterial walls, that the arterial walls are impervious, and that the inlet and outlet of the artery segment have no axial displacement. The inlet velocity and outlet pressure are prescribed as indicated in Table 1 and represented mathematically as:

$$\begin{matrix}u {\vert}_{\prod } = ( 0 , 0 ) & [ Equation 9 ] \\ \frac{\partial u}{\partial x} {\vert}_{inlet , outlet} = ( 0 , 0 ) & [ Equation 10 ] \\ u {\vert}_{x = 0} = {u}_{in} = 0.2 m \text{/} s & [ Equation 11 ] \\ p {\vert}_{x = 1} = {p}_{out} = 0.0 {Nm}^{- 2} & [ Equation 12 ]\end{matrix}$$

where u is the inflow velocity vector, pₒᵤₜ is the pressure at the outlet, and Π is the interface between fluid and structure domains.

The viscous stresses are related to the deformation rates for the assumed Newtonian flow, thus:

$$\begin{matrix}{τ}_{xx} = 2 μ \frac{\partial μ}{\partial x} - \frac{2}{3} μ ( ∇ \cdot {V}^{r} ) & [ Equation 13 ] \\ {τ}_{yy} = 2 μ \frac{\partial v}{\partial y} - \frac{2}{3} μ ( ∇ \cdot {V}^{r} ) & [ Equation 14 ] \\ {τ}_{xy} = {τ}_{yx} = μ ( \frac{\partial μ}{\partial y} + \frac{\partial v}{\partial x} ) & [ Equation 15 ]\end{matrix}$$

The numerical methods uses a two-way implicit coupling between the fluid and structure modules. The pressures and velocities obtained from the flow modules are sent to the stress module at every ten iterations at which deformations and stresses are calculated. Then, the deformations are sent back to the flow module, at which the solution is recalculated on the new deformed geometry. Iterations are performed until convergence is obtained. The convergence criterion continues the iterative solution until the calculated difference between the mass inflow and mass outflow rates is negligible. Typically, the ratio of this difference to the prescribed mass inflow rate is less than 0.1%.

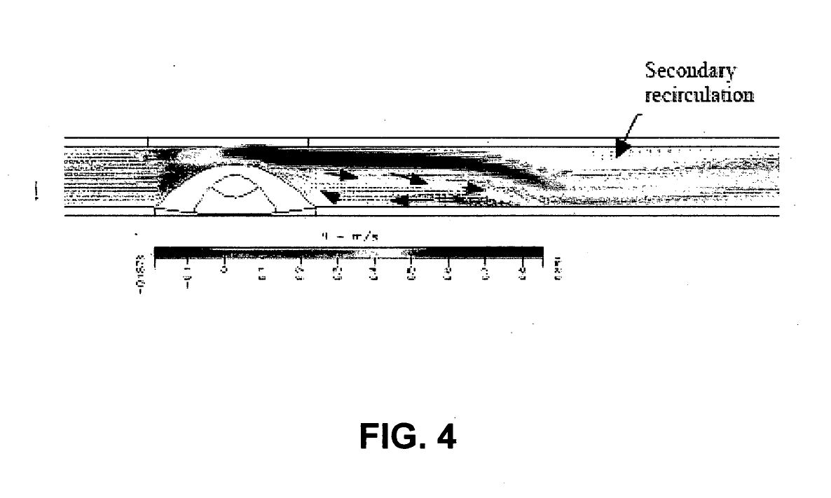

Flow patterns are next predicted for various representative stenosis levels, such as 20%, 40%, and 70%. An example predicted flow pattern for an artery exhibiting a stenosis level of 70% is shown in FIG. 4. As in FIG. 2, an oval-shaped section on the bottom arterial wall represents the plaque or stenosis. Within the stenosis is a smaller oval structure representing the lipid core or pool. As indicated in FIG. 4, the predicted velocity profile is parabolic upstream of the stenosis and the flow becomes fully developed over the 12D length upstream of the stenosis. Then, the velocity increases within the constricted section above the stenosis. The flow rate through the artery is predicted to be at a maximum value ranging from 0.34 m/s for 20% stenosis to 0.85 m/s for 70% stenosis. Notably, the parabolic profile is progressively distorted as the plaque severity increases. As is further indicated in FIG. 4, a small recirculation vortex develops in the lee of the stenosis due to a decrease in pressure in the expanding flow channel and the no-slip condition on the surface, the size and the strength of which increase with the stenosis severity.

For the 70% stenosis case shown in FIG. 4, a second re-circulation vortex develops on the upper surface. The second recirculation vortex occurs for the 70% stenosis case due to the combination of flow momentum and the inertia force created by the first recirculation vortex. In other words, the pull by the first vortex creates a vacuum effect on the opposite upper side of the channel, which is rapidly filled with backward flow to balance the momentum. Notably, the recirculations are important because they impact the deposition of atherogenesis constituents such as low-density lipoproteins (LDLs) in the artery. The deposition is mediated by both the low shear stress and the increased residence time of the constituents in the recirculation zone. The resident time increases with the size of the recirculation vortex.

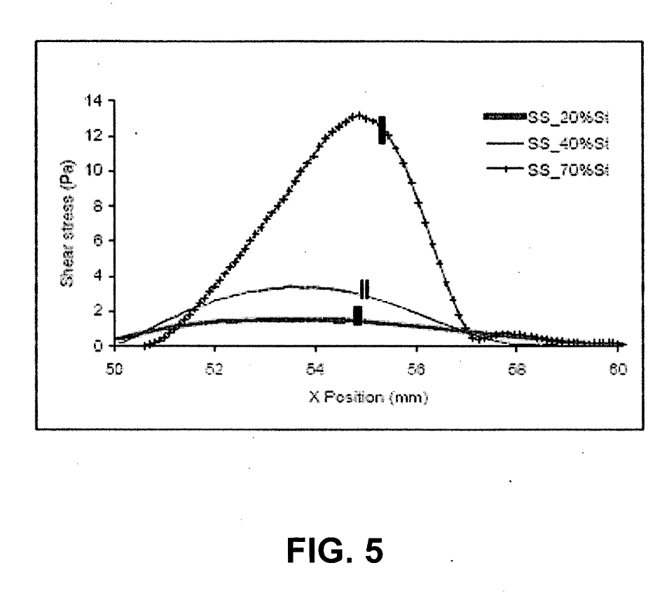

The corresponding distributions of shear stress (SS) for 20%, 40%, and 70% stenosis as a function of horizontal position or “X Position” along the liquid-plaque interface (i.e., from the leading edge of the plaque to its trailing edge) are presented in FIG. 5. The shear stress reflects the effects on the surface of the plaque from blood flow through the artery. In essence, the shear stress reflects the resistance or friction created on the surface of the plaque by blood flow.

The vertical thick lines in FIG. 5 represent the location of the vertical plane (VP) through the stenosis throat. FIG. 5 shows that the endothelial shear stress increases with the stenosis level at the upstream side of the plaque due to the flow acceleration resulting from channel reduction. The wall shear stress rises monotonically to a maximum in the upstream section, and then drops to the lowest value downstream of the VP before oscillating to a fairly constant value. The minimum stress following the drop is located at the re-attachment point downstream of the VP. As illustrated in FIG. 5, the location at which the stress drops from the maximum is quite distinct for the stenosis levels above 40%. As with the pressure distribution, the shear stress increases with stenosis severity, and its maximum occurs just before the VP.

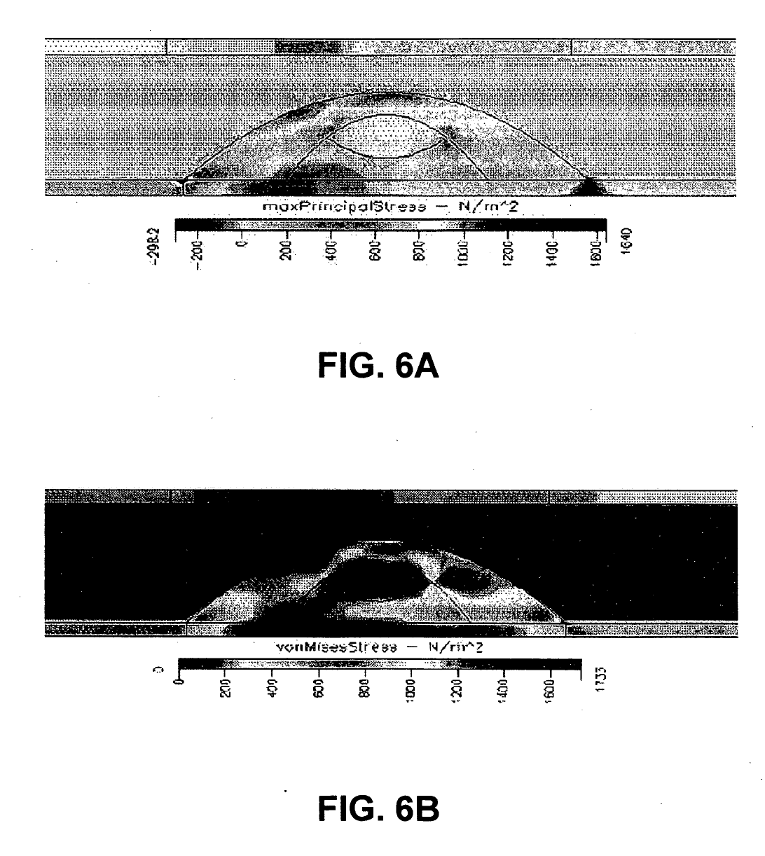

Predicted representative stress contour plots from structural analysis are illustrated in FIGS. 6A and 6B. The plots presented in those figures are the contours of maximum principal stress (FIG. 6A) and Von Mises stress (FIG. 6B) for the 70% stenosis model. Both the maximum principal stress and the Von Mises stress are parameters that pertain to structural stress and, more particularly, the stress within the wall of the plaque (e.g., fibrous cap). The stresses result from the internal resistance of the plaque wall to the pressure imposed by the blood flow through the artery. In cases in which the plaque wall is relatively elastic, the maximum principal stress and the Von Mises stress will be relatively low. In cases in which the plaque wall is relatively plastic, however, those stresses will be relatively high.

FIG. 6A shows that plaque undergoes compressive and extensive stress predominantly in the upstream section while, under the same hemodynamic conditions, FIG. 6B shows coexistence of low and high Von Mises stress bands in the stenosis. As expected, the lowest Von Mises stress contours are located in the lipid pool and areas scattered adjacent to the lipid pool. FIGS. 6A and 6B illustrate the effect of lipid pool on biomechanical stress distribution in the stenotic plaque.

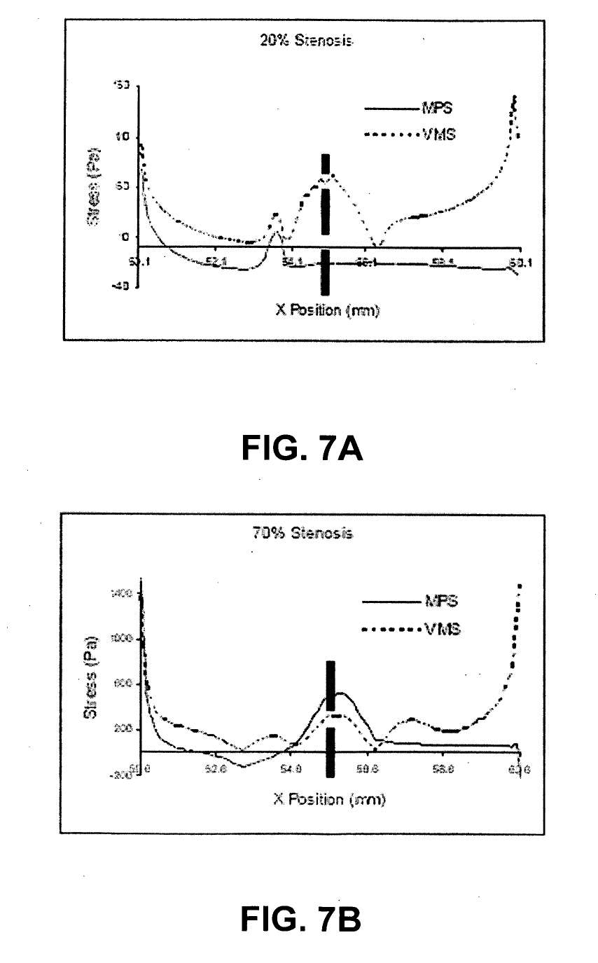

FIGS. 7A and 7B show the maximum principal stress (MPS) and Von Mises stress (VMS) for 20% and 70% stenosis levels, respectively, as a function of horizontal position (“X Position”) adjacent the liquid-plaque interface. In the case of FIGS. 7A and 7B, the stress values are obtained from points within the fibrous cap located just below the outer surface of the plaque to fully account for the structural effects. The vertical axis represents the predicted structural stress obtained in N/m². The thick vertical line in FIGS. 7A and 7B represents the location of the VP passing through the stenosis throat.

The results shown in FIGS. 7A and 7B indicate that within the fibrous cap the MPS starts with high positive values at the proximal end of the stenosis and subsequently drops rapidly to negative values. The initial high values are due to stress continuity between the upstream disease-free arterial wall and the diseased segment. The incoming flow compresses the plaque proximally while the upstream wall segment is under tension. That compression produces the observed negative MPS. Peaks of MPS extension are also evident in the model. The main MPS peak for 70% stenosis is located on the VP, and upstream of the VP for 20% stenosis. This trend is due to the lipid pool reaction to the external compression. The MPS increases with stenosis severity on the VP due to the low pressure above the plaque. Specifically, the plaque sustains important compression on its upstream side and deforms on its top where there is less resistance in order to balance the surrounding forces. The drop in the MPS curve at the end of the vessel is associated with the compression of the disease-free artery wall distal of the stenosis.

The VMS curves show three consecutive peaks: one on each side of the VP and one on the VP. The peaks on both sides of the VP increase with the stenosis severity while the peak on the VP is relatively high for 20% stenosis (70 N/m²), decreases (to 25 N/m²) for 40% stenosis, and significantly rises (up to ˜350 N/m²) for 70% stenosis.

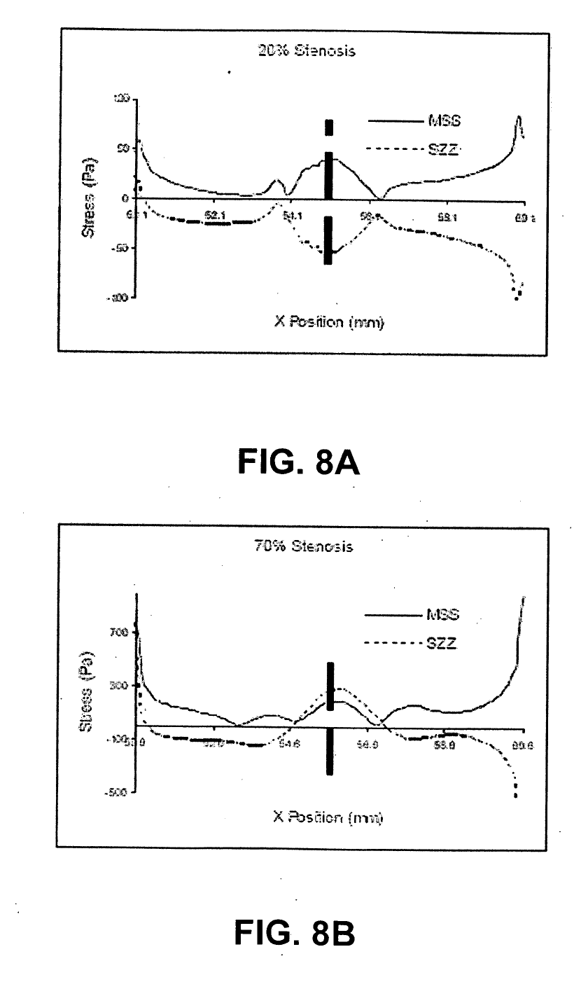

The maximum shear stress (MSS) and circumferential stress (SZZ) for different stenosis levels 20% and 70% are shown in FIGS. 8A and 8B, respectively. The MSS is intended to describe the stress on the planes 45 away from the MPS plane, where the structural shear stress is maximal. The SZZ describes the stress in the direction perpendicular to the model.

The results of FIGS. 8A and 8B indicate that as for MPS, the SZZ curve starts with high positive values and then decreases to negative values. Like the MPS, the positive SZZ values are due to stress continuity between the upstream disease-free arterial wall and the diseased segment. Negative SZZ values are compressive stresses due to the internal pressure obliquely distributed over the diseased segment unlike the case on the disease-free segments where they are radial. On both sides upstream and downstream of the diseased segment, SZZ acts in opposite directions. A peak of SZZ extension identified with positive value is also observed in the graph for the 70% stenosis level (FIG. 8B). It is important to note that similar to the MPS curve, SZZ rises with stenosis severity on the VP.

Similar to that shown above in relation to the VMS, the MSS curve exhibits three consecutive peaks, one on each side of the VP and one on the VP. The peaks on both sides of the VP increase with the stenosis severity, while the peak on the VP is relatively high for mild stenosis (20% stenosis), decreases for moderate stenosis (30%-50% stenosis) (not shown), and significantly rises for severe stenosis (70% stenosis).

In the discussion of FIGS. 2-8, stress data was obtained through mathematical modeling. As noted in the foregoing, however, such stress data can be obtained through empirical testing and/or physical modeling. In such a case, real-world stress data can be collected and can then used to generate the FSI indices.

Generation of Stress Ratios

The stress values described in the foregoing can be used to generate stress ratios that, in turn, can be used to generate FSI indices helpful in characterizing plaque potential to rupture. In at least some cases, the stress ratios comprise both a flow-related component (e.g., shear stress) and a structure-related component (e.g., maximum principal stress, Von Mises stress) given that flow and structure interact in the vascular system.

Considered first are stress ratios R₁ and R₂, where R₁ is the endothelial (wall) shear stress normalized by the maximum principal stress (SS/MPS) and R₂ is the wall shear stress normalized by the Von Mises stress (SS/VMS). The choice of normalizing the shear stress by structural stresses is based upon three reasons. The first reason is the successive compression and extension of structural stress distribution in the plaque as observed in the foregoing. Second, several studies have shown that both shear stress and structural stress play important roles in plaque disruption. The third reason is analogy to the mechanism of buckling in material failure studies with internal pressure in the vessel model related to compressive pressure in the buckled material, and shear stress in the vessel related to perturbation (transverse force) in the material.

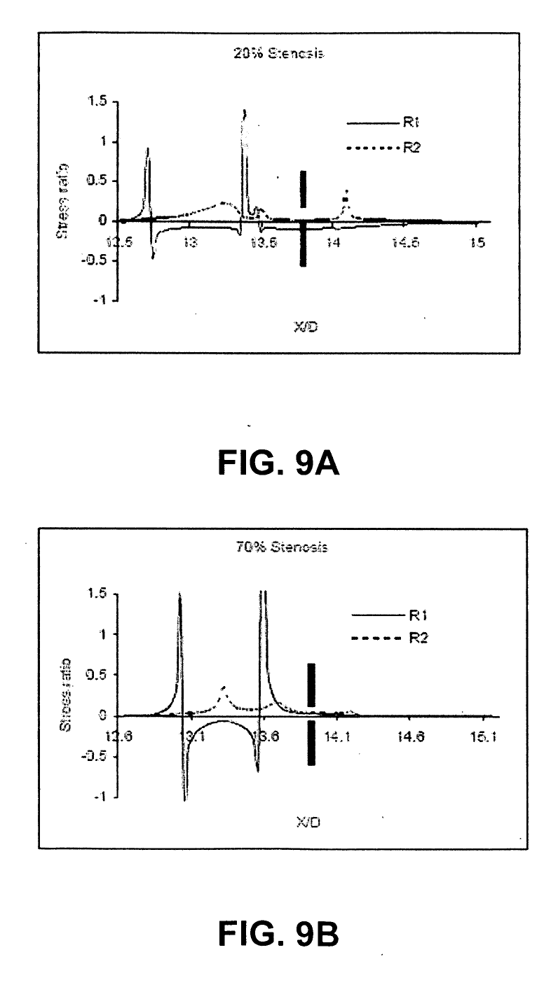

FIGS. 9A and 9B show the distributions of the stress ratios R₁ and R₂ for various values of X/D at 20% and 70% stenosis levels, respectively. The values for the dimensionless “distance” X/D are obtained from the X position along the plaque and the nominal diameter of the artery (i.e., Dᵢ in FIG. 2). As illustrated in FIG. 9A, R₁ has multiple positive and negative peaks. The peaks are located where R₁ is infinite (discontinuous). Such a result is expected since the maximum principal stress (MPS) is zero at those locations. R₁ is negative between the two infinities prior to the VP due to the compressive MPS. Between the peak prior to and on the VP, R₁ is low and positive for moderate stenosis (not shown) and severe stenosis (70%), but remains negative for mild stenosis (20%).

Turning to FIG. 9B, R₂ has two peaks upstream of the VP and one downstream. The first peak is significant because it occurs on the shoulder where plaques are most likely to rupture, its base is larger than the others, and it varies with the stenosis severity. Notably, the R₂ changes with the stenosis level at the location of the first peak of R₁.

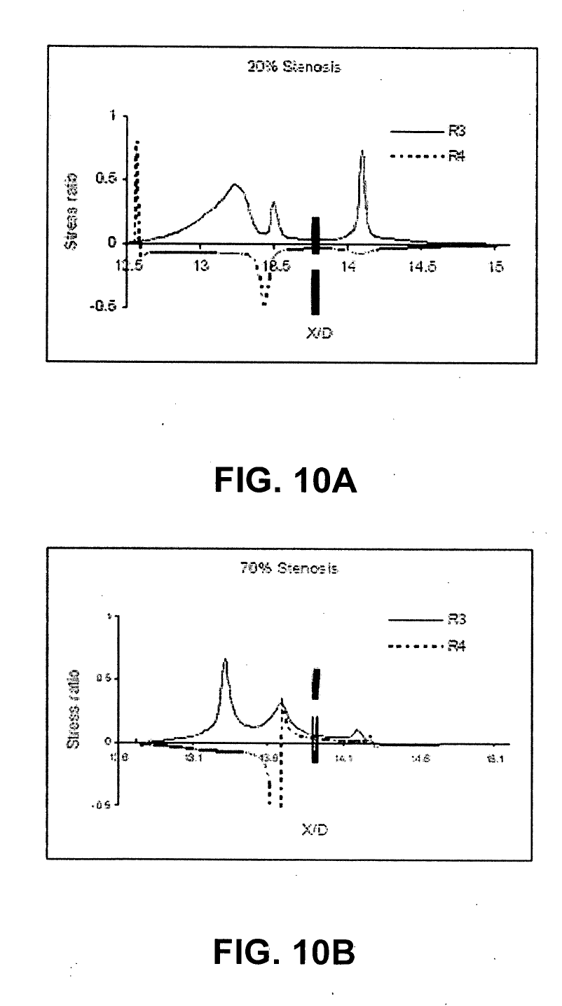

FIGS. 10A and 10B illustrate the behavior of two further stress ratios, R₃ and R₄. R₃ is the ratio of wall shear stress to maximum shear stress (SS/MSS), and R₄ is the ratio of wall shear stress to circumferential stress (SS/SZZ). In FIGS. 10A and 10B, the ratio distributions are presented for 20% and 70% stenosis levels, respectively.

The R₃ curves have similarities to R₂ curves and the characteristics cited previously for R₂ can be applied to R₃. In addition, at the location of the first peak of R₃, R₄ changes with the stenosis level.

As with R₁, R₄ exhibits multiple positive and negative peaks. The peaks are located where R₄ is infinite (discontinuous). That result is expected because the circumferential stress (SZZ) is zero at these locations. Between the two R₄ infinities prior to the VP, R₄ is negative due to the compressive SZZ. At the vicinity of the VP, R₄ remains almost unchanged and close to zero. After the VP, R₄ for moderate (40%) (not shown) and severe (70%) stenosis levels becomes discontinuous again and changes sign at approximately ⅓ the distance from the base of the lesion, downstream of the VP.

Generation of FSI Indices

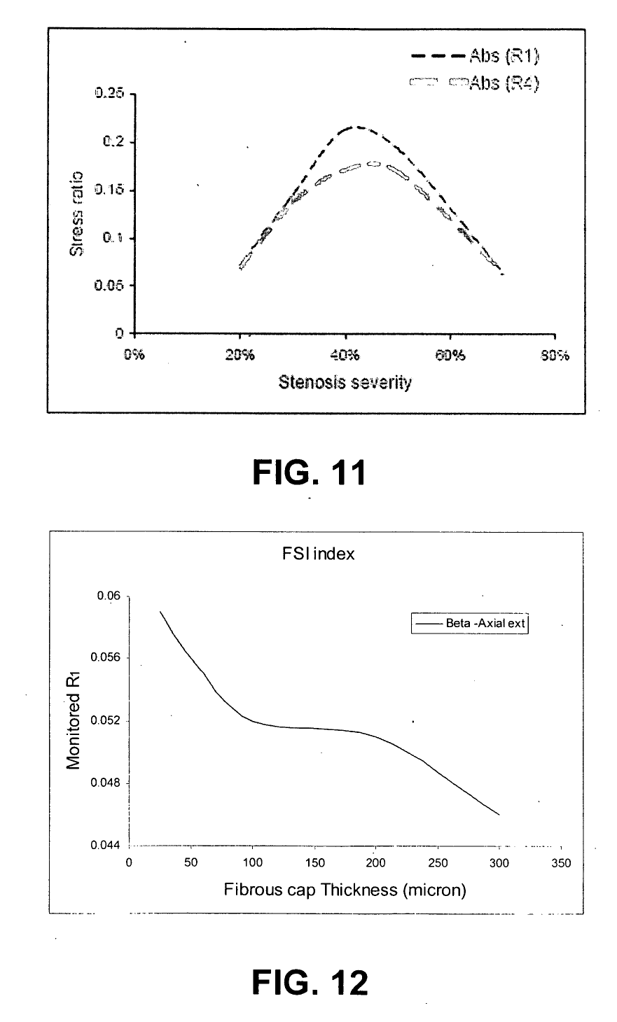

Once stress ratios have been generated, one or more FSI indices are created from the stress ratios relative to one or more plaque characteristics. FIG. 11 illustrates two such indices. The first index is an R₁ index identified as the “Abs (R1)” curve. That curve plots the R₁ stress ratio data obtained for stenosis levels of 20%, 30%, 40%, 50%, and 70% at the point upstream of the VP at which R₂ tends to infinity. That location coincides with the leading shoulder of the plaque, the point at which plaque rupture most often occurs. The second index shown in FIG. 11 is the R₄ index, which is identified as the “Abs (R4)” curve. That curve plots R₄ stress ratio data obtained for stenosis levels of 20%, 30%, 40%, 50%, and 70% at the point upstream of the VP at which R₃ tends to infinity. The curves can be mathematically defined as follows:

$$\begin{matrix}{R}_{1} = \vert {R}_{1} \{ {( X / D )}_{{R 2}_{Max}} \} \vert & [ Equation 16 ] \\ {R}_{4} = \vert {R}_{4} \{ {( X / D )}_{{R 3}_{Max}} \} \vert & [ Equation 17 ]\end{matrix}$$

FIG. 11 shows that the two indices exhibit the same trends, but the R₁ index is consistently larger than the R₄ index. The indices are small for both mild (e.g., 20%) and severe (e.g., 70%) stenosis, indicating a lesser likelihood of plaque rupture at those levels of stenosis. Significantly, the indices reach a maximum between the extreme stenosis levels at approximately 40%-45% stenosis, indicating a higher likelihood of plaque rupture. That indication is consistent with medical observations as to plaque rupture.

As a consequence of FIG. 11, either R₁ or R₄ index could be used to characterize plaque potential to rupture since the results are qualitatively similar. In the following discussion, only R1 is discussed to illustrate application of the FSI concept.

FIGS. 12-14 illustrate further FSI indices. In FIG. 12, R₁ is plotted as a function of fibrous cap thickness in microns (μm). As is apparent from that figure, the index decreases as the fibrous cap thickness increases. That result implies that, consistent with clinical studies, thinner fibrous caps are more prone to rupture.

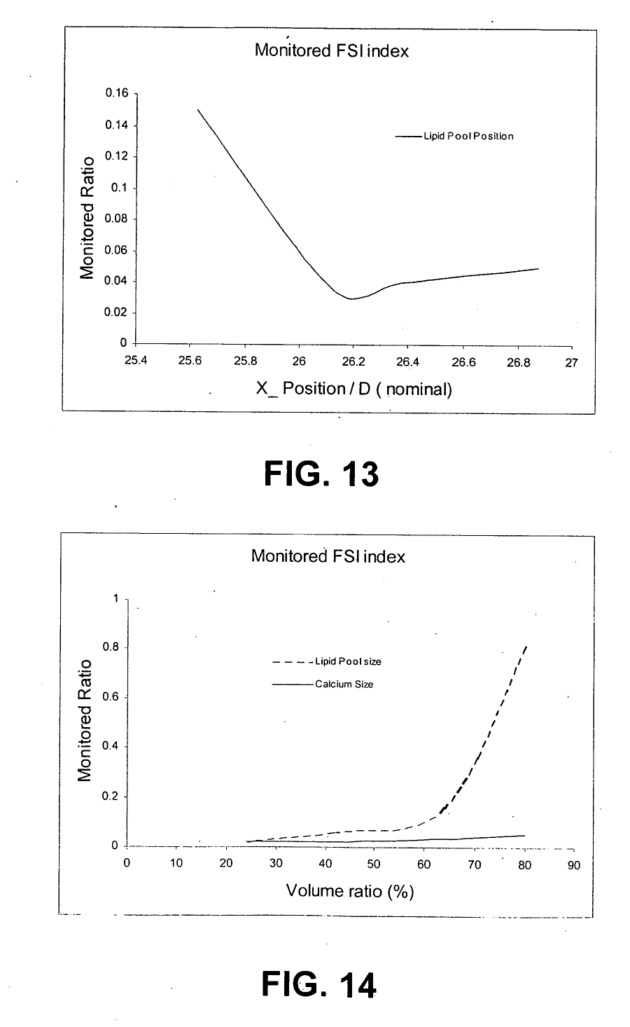

In FIG. 13, R₁ is plotted as a function of lipid pool position within the plaque from the leading to the trailing edge of the stenosis. As can be seen from FIG. 13, the index rapidly decreases as the lipid location shifts from the leading edge to the trailing edge of the plaque. That implies that the lipid pool has a greater impact on plaque rupture when it is located close to the fibrous cap shoulders, particularly the leading fibrous cap shoulder.

In FIG. 14, R₁ is separately plotted as a function of lipid pool volume and calcium volume relative to the total plaque volume. The results of FIG. 14 imply that the likelihood of plaque rupture sharply increases with an increase in the ratio of lipid pool volume to total plaque volume, but increases less so for increases in the ratio of calcium volume to total plaque volume. In addition, the impact of lipid pool on R1, and hence plaque rupture potential, increases dramatically when the relative lipid volume to plaque volume exceeds 60%. This finding is consistent with medical observations.

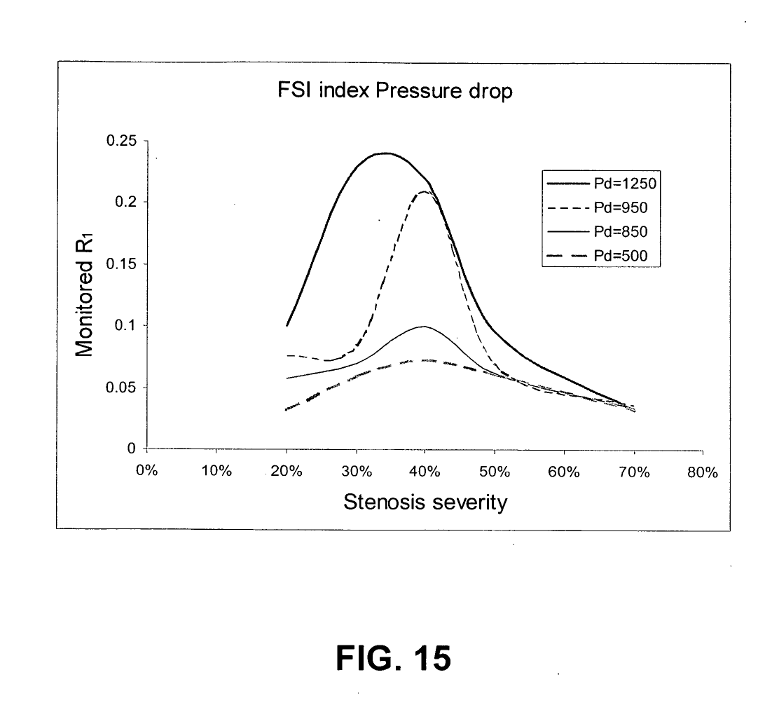

As also described above, multiple FSI indices can be generated relative to test subject or patient type. FIG. 15 provides an example of such FSI indices. More particularly, FIG. 15 illustrates FSI indices that plot the R1 stress ratio data versus stenosis level, with each separate index pertaining to a different blood pressure level. The different blood pressure levels are represented by different pressure drops across the modeled artery (see, e.g., FIG. 2). The pressure drop is a measure of inlet pressure minus outlet pressure. If the outlet pressure is kept constant, the larger the pressure drop, the higher the average blood pressure of the patient. With FSI indices such as those of FIG. 15, a particular FSI index can be selected for a patient under evaluation based upon his or her blood pressure. Notably, FIG. 15 shows that the peak FSI progressively shifts towards lower stenosis rates at higher blood pressures. In effect, the results indicate that high blood pressure may render otherwise benign or mild plaques unstable and vulnerable to rupture. Conversely, all plaques become more vulnerable to rupture as the blood pressure increases.

Determination of Plaque Vulnerability

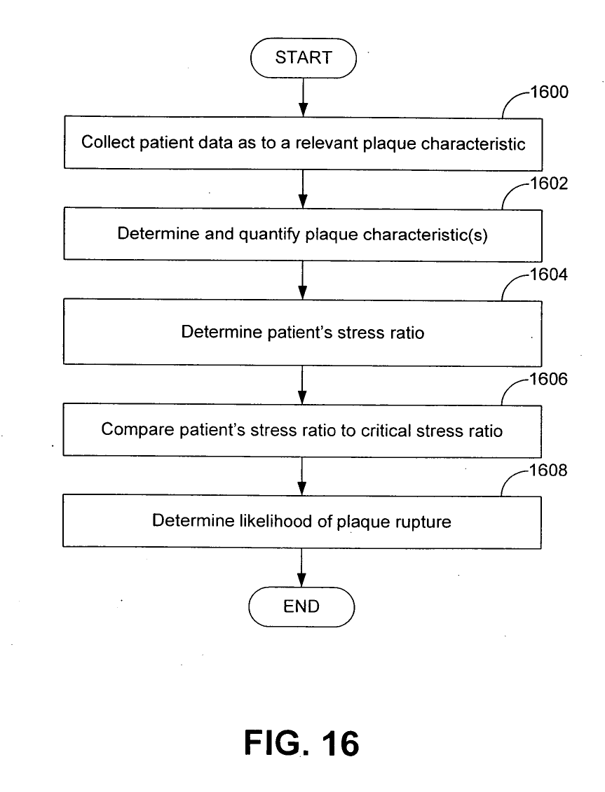

Once an FSI index has been generated, it can be used as an aid in gauging plaque vulnerability and therefore predicting plaque rupture. FIG. 16 illustrates an embodiment of such a method. Beginning with block 1600, data is collected from a patient under evaluation as to a relevant plaque characteristics and patient data. The relevant data includes stenosis rate, fibrous cap thickness, lipid pool size, lipid pool location within the plaque, calcium deposit, and patient blood pressure. The characteristic may be determined by the nature of the FSI index that is used. The dominant characteristic may be deduced from patient risk potential (legacy data mining), optimization studies, and statistical analysis from the combination of parameters. For example, if the FSI index comprises the R1 index of FIG. 11, the plaque characteristic may be stenosis level as that index is a function of stenosis level. If the FSI index is a function of another plaque characteristic, such as fibrous cap thickness, data may be collected as to that characteristic. In some embodiments, the data can be collected through imaging of a diseased artery of the patient. For example, one or more of ultrasound, magnetic resonance imaging (MRI), optical coherence tomography (OCT), or fluorescence spectroscopy can be used to make determinations as to the relevant plaque characteristic. With that data, the patient's particular plaque characteristic(s) can be determined and quantified, as indicated in block 1602.

Once the patient's plaque characteristic(s) has or have been determined, the characteristic(s) can be used to determine the patient's stress ratio, as indicated in block 1604. For example, if the R1 index of FIG. 11 is used as the FSI index and the level of stenosis for the patient is determined to be 30%, the patient's R1 stress ratio can be determined to be approximately 0.15. This value may change depending on advances in computational methods, structural models of plaque and arterial walls, and other models on which the determination of R₁ values of the type presented in FIG. 11 depend.

Next, the patient's stress ratio is compared to a critical stress ratio, as indicated in block 1606. The critical stress ratio is a ratio over which plaque rupture is deemed likely. Therefore, the critical stress ratio can be considered as a threshold value that is used to make the plaque vulnerability determination. In some embodiments, the critical stress ratio will be near the peak of the applied FSI index. The critical stress ratio can either be determined based upon mathematical approximation or upon empirical data, such as test data from animal subject up through plaque rupture.

Through comparison of the patient's stress ratio and the critical stress ratio, the likelihood of plaque rupture can be determined, as indicated in block 1608. Such a determination can, in some embodiments, be based upon the comparison alone. For example, if the patient's stress ratio is 0.15 and the critical stress ratio is 0.13, it may be assumed that plaque rupture is likely and appropriate steps may be taken, such as immediate surgery. In other embodiments, the determination can be made by a physician in view of other relevant factors. For example, if the patient's stress ratio is just above the critical stress ratio but the nature of the lipid core and/or the fibrous cap indicates a reduced likelihood of rupture, the physician may decide that immediate surgery is not required.

As described in the foregoing, the FSI index that is used may depend upon the type of patient that is being evaluated. For example, a first FSI index may be used for normal blood pressure patients, a second FSI index used for low blood pressure patients, and a third FSI index used for high blood pressure patients.

In some embodiments, the various FSI indices will be determined, and statistical analysis coupled with patent historical data will be used to chose the dominant characteristic for determining the stress ratio for comparison with the critical index.

Example Apparatus

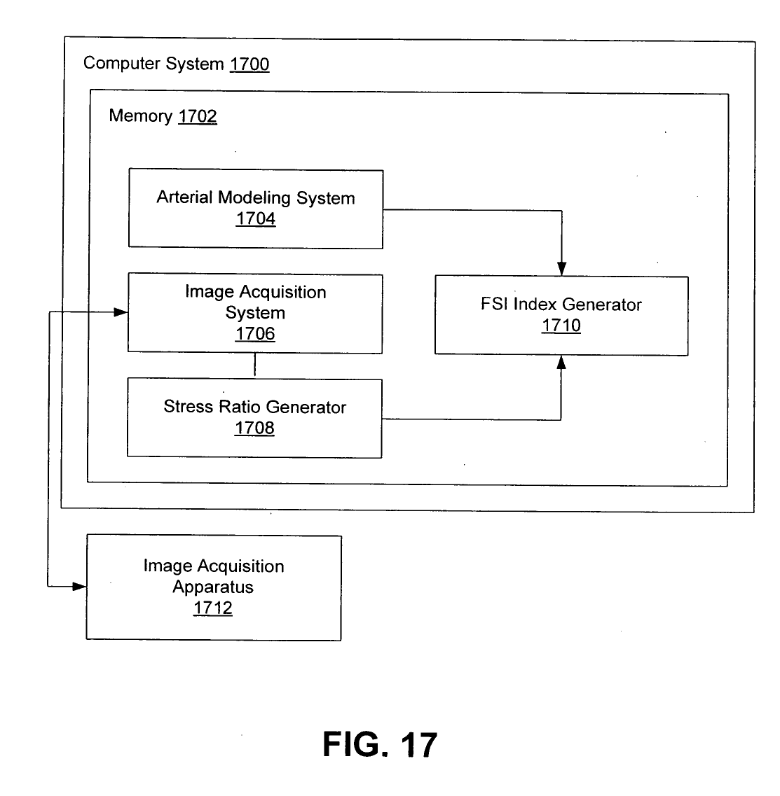

FIG. 17 illustrates a computer system 1700 that can be used to generate FSI indices. The system 1700 includes a computer-readable medium in the form of computer memory 1702. By way of example, the computer system 1700 comprises a desktop, laptop, or server computer that includes the computing and processing power necessary to conduct the data collection and manipulation described in the following. Although the computer system 1700 can comprise a single computer, the system can, alternatively, comprise two or more such computers. For example, multiple networked computers can be used, if desired. Also by way of example, the memory 1702 can comprise a combination of volatile and non-volatile memory components. For instance, the memory 1702 may comprise one or more hard disks and one or more random access memory (RAM) components. In addition, the memory 1702 can comprise read-only memory (e.g., Flash memory) and one or more removable memory components, such as a floppy disk, a CD-ROM, or a memory card.

Stored within memory 1702 is an arterial modeling system 1704, an image acquisition system 1706, a stress ratio generator 1708, and an FSI index generator 1710. The arterial modeling system 1704 comprises the various logic that is configured to generate a model of a diseased artery and mathematically generate stress data that can be used to compute stress ratio data. The image acquisition system 1706 can be coupled to imaging apparatus 1712 that is used to capture image data of test subjects such that the image data can be provided to the stress ratio generator 1708 to identify the stresses affecting a diseased artery and compute the stress ratios associated therewith. The FSI index generator 1710 is configured to generate FSI indices relative to stress ratio data provided by either the arterial modeling system 1704 or by the stress ratio generator 1708. As described above, the FSI indices can then be used to determine plaque rupture potential in relation to a patient under evaluation.

1. A method for determining plaque rupture potential , the method comprising:
obtaining stress values for a diseased artery;
determining stress ratios from the obtained stress values; and
generating a flow-structure interaction index based upon the stress ratios as a function of a given plaque characteristic.

2. The method of claim 1, wherein obtaining stress values comprises obtaining stress values through mathematical modeling and mathematical computation of the stress values.

3. The method of claim 1, wherein obtaining stress values comprises obtaining stress values through subject testing and determination of the stress values.

4. The method of claim 1, wherein obtaining stress values comprises obtaining stress values through physical modeling and measurement of the stress values.

5. The method of claim 1, wherein obtaining stress values comprises obtaining flow-related stress values and structure-related stress values.

6. The method of claim 5, wherein obtaining flow-related stress values comprises obtaining shear stress values at a plaque-blood interface and wherein obtaining structure-related stress values comprises obtaining structural stress values within a fibrous cap of a plaque.

7. The method of claim 6, wherein the structural stress values comprises one of maximum principal stress values or Von Mises stress values.

8. The method of claim 1, wherein determining stress ratios comprises determining ratios between flow-related stress values and structure-related stress values.

9. The method of claim 8, wherein determining ratios between flow-related stress values and structure-related stress values comprises determining ratios between shear stress values at a plaque-blood interface and structural stress values within a fibrous cap of a plaque.

10. The method of claim 1, wherein generating a flow-structure interaction index comprises calculating the stress ratios as a function of stenosis level.

11. The method of claim 1, wherein generating a flow-structure interaction index comprises calculating the stress ratios as a function of fibrous cap thickness.

12. The method of claim 1, wherein generating a flow-structure interaction index comprises calculating the stress ratios as a function of lipid pool position.

13. The method of claim 1, wherein generating a flow-structure interaction index comprises calculating the stress ratios as a function of a ratio of lipid pool volume versus total plaque volume.

14. The method of claim 1, wherein generating a flow-structure interaction index comprises generating multiple flow-structure interaction indices for various levels of blood pressure.

15. The method of claim 1, further comprising determining a plaque characteristic of a patient and determining a patient stress ratio.

16. The method of claim 15, further comprising comparing the patient stress ratio with a critical stress ratio and, if the patient stress ratio exceeds the critical stress ratio, determining that a plaque of the patient is vulnerable to rupture.

17. A method for generating a flow-structure interaction index, the method comprising:
determining shear stresses that act upon an arterial plaque due to blood flow;
determining structural stresses within a fibrous cap of the arterial plaque resulting from internal resistance within the fibrous cap due to pressure imposed by the blood flow;
calculating stress ratios that comprise ratios of the shear stresses and the structural stresses; and
calculating a flow-structure interaction index that comprises a relation of stress ratio as a function of a given plaque characteristic.

18. The method of claim 17, wherein calculating a flow-structure interaction index comprises calculating stress ratio as a function of one of stenosis level, fibrous cap thickness, lipid pool position, or a ratio of lipid pool volume versus total plaque volume.

19. The method of claim 17, wherein calculating a flow-structure interaction index comprises generating multiple flow-structure interaction indices for various levels of blood pressure.

20. A method of determining plaque rupture potential, the method comprising:
determining a plaque characteristic of a patient under evaluation;
using the plaque characteristic to determine a patient stress ratio through reference to a flow-structure interaction index that comprises a relation of stress ratio as a function of the plaque characteristic, the stress ratio comprising a ratio of shear stress and structural stress;
comparing the patient stress ratio to a critical stress ratio over which a plaque is vulnerable to rupture; and
determining whether the plaque is vulnerable to rupture relative to the comparison.

21. The method of claim 20, wherein the plaque characteristic comprises of one of stenosis level, fibrous cap thickness, lipid pool position, or a ratio of lipid pool volume versus total plaque volume.

22. The method of claim 20, wherein the flow-structure interaction index has been calculated relative to a given blood pressure level.

23. A computer-readable medium comprising:
logic configured to determine shear stresses that act upon an arterial plaque due to blood flow;
logic configured to determine structural stresses within a fibrous cap of the arterial plaque resulting from internal resistance within the fibrous cap due to pressure imposed by the blood flow;
logic configured to calculate stress ratios that comprise ratios of the shear stresses and the structural stresses; and
logic configured to calculate a flow-structure interaction index that comprises a relation of stress ratio as a function of a given plaque characteristic.

24. The method of claim 23, wherein the logic configured to calculate a flow-structure interaction index comprises logic configured to calculate stress ratio as a function of one of stenosis level, fibrous cap thickness, lipid pool position, or a ratio of lipid pool volume versus total plaque volume.

25. The method of claim 23, wherein the logic configured to calculate a flow-structure interaction index comprises logic configured to generate multiple flow-structure interaction indices for various levels of blood pressure.

26. A plaque vulnerability determination system, the system comprising:
means for determining a plaque characteristic of a patient under evaluation; and
means for determining a patient stress ratio, the stress ratio comprising a ratio of shear stress and structural stress that act upon and in a plaque of the patient.

27. The system of claim 26, wherein the plaque characteristic comprises of one of stenosis level, fibrous cap thickness, lipid pool position, or a ratio of lipid pool volume versus total plaque volume.

28. The system of claim 26, wherein the means for determining a patient stress ratio comprise a flow-structure interaction index that comprises a relation of stress ratio as a function of the plaque characteristic.

29. The system of claim 26, further comprising means for comparing the patient stress ratio to a critical stress ratio over which a plaque is vulnerable to rupture.

30. The system of claim 29, further comprising means for determining whether the plaque is vulnerable to rupture relative to the comparison.
